(19) United States
(12) Patent Application Publication
(10) Pub. No.: US 2025/0078974 A1
(43) Pub. Date: Mar. 6, 2025

(54) PROVIDING PRIORITIZED PRECISION TREATMENT RECOMMENDATIONS

(71) Applicant: Clarified Precision Medicine, LLC, Tampa, FL (US)

(72) Inventors: Daniel ROTROFF, Pepper Pike, OH (US); Jody SIMON, Tampa, FL (US); Howard MCLEOD, Tampa, FL (US); Lincoln NADAULD, Santa Clara, UT (US); Derrick HASLEM, Saint George, UT (US); Terence RHODES, Washington, UT (US); Will CORUM, Nicholasville, KY (US); Neil MASON, Land o' Lakes, FL (US)

(21) Appl. No.: 18/290,143

(22) PCT Filed: May 10, 2022
(86) PCT No.: PCT/US22/28611

§ 371 Date: Nov. 9, 2023

Related U.S. Application Data
(60) Provisional application No. 63/186,768, filed on May 10, 2021.

(51) Int. Cl. G16H 20/10; G06N 5/022

(52) U.S. Cl. CPC G16H 20/10; G06N 5/022

(57) ABSTRACT

A machine learning-based system, and corresponding methods of use, prioritize therapeutic regimens based on genetic variations to provide ranked treatment recommendations.

### CROSS-REFERENCE TO RELATED APPLICATIONS

This application claims the benefit of priority to U.S. Provisional Application No. 63/186,768, filed May 10, 2021, the contents of which are hereby incorporated by reference in their entirety and for all purposes.

### BACKGROUND OF THE INVENTION

Research has shown that precision oncology not only improves outcomes for cancer patients, but results in lower average per-week healthcare costs, resource utilization, and end-of-life costs. However, the large number of combinations of genetic mutations linked to available targeted therapies presents a substantial challenge for healthcare providers who try to keep up with innovations in this field. For example, The Cancer Genome Atlas (TCGA) has reported more than 3.4 million somatic genetic mutations in 23,535 genes across 67 different cancer sites. See Weinstein J N et al., The cancer genome atlas pan-cancer analysis project, Nature Genetics, October 2013; 45(10):1113-20.

In addition to targeted therapies, precision medicine approaches allowing germline driven decisions to reduce treatment related toxicities and adverse outcomes are becoming increasingly important for the clinical management of patients. Currently, there are more than 290,000 germline genetic variants across 9945 genes considered to have potentially pathogenic or drug response effects, according to the National Center for Biotechnology Information's ClinVar database. See Landrum M J et al, ClinVar: improving access to variant interpretations and supporting evidence, Nucleic acids research, Jan. 4, 2018; 46(D1):D1062-7.

Still further, patients can have multiple actionable genetic mutations triggering multiple possible FDA approved treatments from which a provider can choose. But electing among the myriad options to prioritize possible patient treatments requires substantial training and domain knowledge that is not widely available. In larger institutions, it may be possible to get access to an entity such as a Molecular Tumor Board. See Mangat P K et al., Rationale and design of the targeted agent and profiling utilization registry study, JCO precision oncology, July 2018; 2:1-4. Unfortunately, however, many healthcare providers lack access to such an entity, and are often forced by time and cost constraints to make treatment decisions based on incomplete information.

### SUMMARY OF THE INVENTION

Aspects of the invention provide a machine learning-based system that tracks expert driven treatment recommendations by genetic variation and cancer type, enabling discernment of treatment recommendations made for genetic variation in the context of competing available FDA approved treatment options. With such a system, optimized treatment recommendations, made by expert-driven consensus, can be made rapidly and widely available to healthcare providers for their patients.

In one aspect, methods of generating a prioritized precision treatment recommendation are provided, comprising: receiving genetic sequence data for said patient comprising at least one genetic mutation; applying said patient-specific genetic sequence data comprising said at least one genetic mutation identified in one or more samples of a patient, to a machine learning system trained on a knowledge base comprising a plurality of genetic mutations across a plurality of genes to map said genetic sequence data to said knowledgebase; said knowledgebase mapping said plurality of genetic mutations to efficacy profiles for therapeutic regimens for the disease, and/or further mapping said genetic mutations to drug-induced toxicities selected from the group consisting of cardiotoxicity, neurotoxicity, hematological toxicity, and anesthesia toxicity; determining, by the machine learning system, a plurality of therapeutic regimens, which may be actionable as a treatment recommendation for said disease for said patient based on one or more of treatment response, treatment resistance, or treatment toxicity; and prioritizing, by said machine learning system, the therapeutic regimens to provide a plurality of ranked treatment recommendations for said disease for said patient as determined by the machine learning system.

The at least one genetic mutation may be somatic and/or germline. In some embodiments, each mutation is mapped to a drug and provided a ranking relative to other genes. The patient-specific genetic sequence data may include identified genetic mutations upon receipt, or these may be separately identified prior to mapping.

In some embodiments, the method further comprises reviewing, by an expert, the ranked treatment recommendations and, responsive to a determination that the ranked treatment recommendations should be reordered or changed, providing a revised set of ranked treatment recommendations. In some embodiments, the knowledgebase is updated based on the revised set of ranked treatment recommendations.

In some embodiments, the method further comprises communicating the ranked treatment recommendations for said disease for said patient to the patient and/or to the patient's caregiver. In some embodiments, the ranked treatment recommendations may comprise off-label uses and/or clinical trials. In some embodiments, the ranked treatment recommendations further comprise supporting literature citations. In some embodiments, the knowledge base further maps said genetic mutations to supportive care pharmacogenomics, and the treatment recommendations further comprise palliative care.

In exemplary embodiments wherein the disease is cancer, the genetic sequence data may comprise tumor panel sequencing data from at least one tumor sample from said patient, and the machine learning system is trained on a knowledge base comprising a plurality of genetic mutations across a plurality of genes in a plurality of tumor types from a plurality of individuals and a plurality of treatments. The plurality of genetic mutations may comprise sequence variants with known functional effects and/or sequence variants with unknown clinical significance.

In another aspect, methods of treating a disease in a patient in need thereof are provided, comprising: receiving genetic sequence data for said patient comprising at least one genetic mutation; applying genetic sequence data comprising said at least one genetic mutation identified in one or more samples of a patient, to a machine learning system trained on a knowledgebase comprising a plurality of genetic mutations across a plurality of genes to map said genetic sequence data to said knowledge base; said knowledgebase mapping said plurality of genetic mutations to efficacy profiles for therapeutic regimens for the disease, and/or further mapping said genetic mutations to drug-induced toxicities selected from the group consisting of cardiotoxicity, neurotoxicity, hematological toxicity, and anesthesia toxicity; determining, by the machine learning system, a plurality of therapeutic regimens, which may be actionable as a treatment recommendation for said disease for said patient based on one or more of treatment response, treatment resistance, or treatment toxicity; prioritizing, by said machine learning system, the therapeutic regimens to provide a plurality of ranked treatment recommendations for said disease for said patient as determined by the machine learning system; communicating the ranked treatment recommendations for said disease for said patient to the patient's caregiver; and administering, by said caregiver, at least one of the ranked treatment recommendations.

The at least one genetic mutation may be somatic and/or germline. In some embodiments, each mutation is mapped to a drug and provided a ranking relative to other genes. The patient-specific genetic sequence data may include identified genetic mutations upon receipt, or these may be separately identified prior to mapping.

In some embodiments, the method further comprises reviewing, by an expert, the ranked treatment recommendations and, responsive to a determination that the ranked treatment recommendations should be reordered or changed, providing a revised set of ranked treatment recommendations. In some embodiments, the knowledgebase is updated based on the revised set of ranked treatment recommendations.

In some embodiments, the ranked treatment recommendations may comprise off-label uses and/or clinical trials. In some embodiments, the ranked treatment recommendations further comprise supporting literature citations. In some embodiments, the knowledge base further maps said genetic mutations to supportive care pharmacogenomics, and the treatment recommendations further comprise palliative care.

In exemplary embodiments wherein the disease is cancer, the genetic sequence data may comprise tumor panel sequencing data from at least one tumor sample from said patient, and the machine learning system is trained on a knowledge base comprising a plurality of genetic mutations across a plurality of genes in a plurality of tumor types from a plurality of individuals and a plurality of treatments. The plurality of genetic mutations may comprise sequence variants with known functional effects and/or sequence variants with unknown clinical significance.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 is a high level flow chart of operation according to an embodiment.

FIG. 2 is a high level block diagram of a system according to an embodiment.

FIG. 3 illustrates the generation of ranked treatment recommendations based on a genetic profiling report from a tumor sample.

FIG. 4 illustrates the generation of ranked treatment recommendations based on genomic sequencing data.

FIG. 5 illustrates the generation of ranked treatment recommendations based on genomic sequencing data.

### DETAILED DESCRIPTION OF EMBODIMENTS

### Definitions

For purposes of interpreting this specification, the following definitions will apply, and whenever appropriate, terms used in the singular will also include the plural and vice versa. In the event that any definition set forth conflicts with any document incorporated herein by reference, the definition set forth below shall control. Unless defined otherwise, all technical and scientific terms used herein have the same meaning as commonly understood by one of ordinary skill in the art to which the disclosure pertains.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The terms “patient,” “subject,” “individual,” and the like are used interchangeably herein, and refer to any animal, amenable to the methods described herein. In certain non-limiting embodiments, the patient, subject or individual is a human.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies.

The term “tumor” as used herein, refers to all neoplastic cell growth and proliferation, whether malignant or benign, and all pre-cancerous and cancerous cells and tissues. In some embodiments, a “tumor” is a “cancerous tumor” and comprises one or more cancerous cells. Therefore, in some embodiments, the term “cancer” is equivalent to the term “tumor.”

The term “therapeutic regimen”, as used herein, refers to a dosing regimen whose administration across a relevant population is or is expected to be correlated with a desired or beneficial therapeutic outcome.

The terms “predictive” and “prognostic” as used herein are also interchangeable. In one sense, the methods for prediction or prognostication are to allow the person practicing a predictive/prognostic method as disclosed herein to select patients that are deemed (usually in advance of treatment, but not necessarily) more likely to respond to a therapeutic regimen or treatment.

The term “knowledgebase” or “knowledge-base” as used herein refers to a store of information or data available for making a diagnosis and recommending treatment for a disease e.g., cancer. The knowledgebase comprises information related to a plurality of genetic mutations, including actionable mutations, across a plurality of genes and a plurality of therapeutic regimens which may be actionable as a treatment recommendation for a disease for a particular patient based on one or more of treatment response, treatment resistance, or treatment toxicity. Thus, as used herein a “knowledgebase” maps a plurality of genetic mutations to efficacy profiles for available therapeutic regimens for a disease, and may further map genetic mutations to drug-induced toxicities such as e.g., cardiotoxicity, neurotoxicity, hematological toxicity, and anesthesia toxicity. In some embodiments, the efficacy profiles take into account cancer type. For example, a gene/drug combination may be particularly effective in colorectal cancer, but become down-weighted in place of other gene/drug combinations in lung cancer. Similarly, systematic guidance regarding the efficacy profile for different age/sex/race, can be included if available.

The knowledgebase disclosed herein is constantly updated and ever-expanding. Sources of information include e.g., medical literature and public databases. Furthermore, as treatment recommendations are provided for patients, this information is received by the system and machine learning is used to incorporate this information into the knowledgebase to continuously refine which gene/drug combinations are most likely to be recommended for a patient. Thus, the “knowledgebase” not only stores data, but also learns and stores other knowledge derived from the data. A “knowledgebase” is accessed by the machine learning system to prioritize the therapeutic regimens to provide a plurality of prioritized one or more treatment recommendations for said disease for a patient.

Thus, the computer processing systems, computer-implemented methods, apparatus and/or computer program products described herein can employ hardware and/or software to generate therapeutic regimens that are highly technical in nature, that are not abstract and that cannot be performed as a set of mental acts by a human. For example, the one or more embodiments can perform the lengthy and complex interpretation and analysis of a copious amount of available information to generate optimized therapeutic regimens and determine which genetic mutations from the one or more genetic mutations should be prioritized for a therapeutic regimen. In another example, if a patient has 3 mutations which have 3 different drugs, which one should they take first. Thus, the knowledgebase and machine learning system can provide prioritized treatment options for effective therapy.

The term “actionable genetic mutations” or “actionable mutations” as used herein refers to known variants validated in the peer-reviewed literature, for which a clinically actionable medical intervention, or preventative approach is available. Thus, “actionable genetic mutations” typically have “known functional effects.”

Genetic mutations having “unknown functional effects” include those genetic variations from a standard control for which the phenotype, disease relationship, or functional effect has not been established.

The phrase “provided a ranking relative to other genes” as used herein refers to determining which treatment recommendations should be prioritized for an individual patient. Different genes may have a greater or lesser effect/interactions with different therapies. Some drugs may carry an increased risk of side effects in a particular genetic background or the patient may be resistant to a particular treatment based on their genetics. Accordingly, these factors are taken into consideration when presenting treatment recommendations for an individual patient.

The term “supportive care pharmacogenomics” as used herein refers to supportive care for pain control, depression, anti-platelet/coagulation, etc. including e.g., what drugs may increase risk of side effects or which ones the patient may be resistant or likely to respond to.

The term “palliative care” as used herein refer to supportive services that are intended for both the person facing illness and their loved ones. Palliative care can be provided at any stage of a chronic or serious illness—as well as at the end of life and may include “supportive care pharmacogenomics.”

### General Methods

This disclosure utilizes routine methods in the fields of statistics and machine learning. Basic texts disclosing the general methods and terms statistics and machine learning include e.g., Fawcett, Tom (2006) Pattern Recognition Letters. 27 (8): 861-874; Encyclopedia of Machine Learning and Data Mining, Claude Sammut, and Geoffrey I. Webb, eds. Springer (2017) and The Elements of Statistical Learning: Data Mining, Inference, and Prediction, Trevor Hastie, Robert Tibshirani, and Jerome Friedman, eds. 2nd Edition Springer (2017).

This disclosure also utilizes routine methods in the field of bioinformatics. Basic texts disclosing the general methods and terms in bioinformatics include e.g., Current Protocols in Bioinformatics, Andreas D. Baxevanis and Daniel B. Davison eds. Wiley (2003). This disclosure utilizes routine concepts and techniques in the field of recombinant genetics. Basic texts disclosing the general methods and terms in molecular biology and genetics include e.g., Sambrook et al., Molecular Cloning, a Laboratory Manual, Cold Spring Harbor Press 4th edition (Cold Spring Harbor, N.Y. 2012); Current Protocols in Molecular Biology Volumes 1-3, John Wiley & Sons, Inc. (1994-1998) and periodic updates. General texts disclosing genome sequencing include e.g., Genome Sequencing Technology and Algorithms Kim, S, Tang, H., and Mardis, E. R., eds. Artech House Inc. (2007). Single cell technologies and genome sequencing methods are reviewed e.g., in Picelli, S. (2017) RNA Biol. 14(5): 637-650.

### INTRODUCTION

Aspects of the invention include a knowledgebase that is trained using machine learning techniques to provide prioritized recommendations for treating a specific patient. A physician receiving the prioritized recommendations may alter the order, or may substitute one or more treatments for one or more of the recommendations. The knowledgebase may be updated (trained further) using the altered order and/or substituted treatment(s).

In an embodiment, a knowledgebase contains genetic variation/mutations mapped to one or more drug-induced toxicities including cardiotoxicity, neurotoxicity, hematological toxicity, anesthesia toxicity, and also to supportive care pharmacogenomics (i.e., drug response). The knowledgebase also may map actionable somatic mutations to drugs that are likely to be effective or those that may be resistant. Treatments may be ranked based on preference and cancer type. In an embodiment, the knowledge base may be curated from publicly available databases and expert review.

In an embodiment, germline or somatic sequencing may be performed. In some instances, this sequencing may be performed by an outside vendor (e.g., Foundation Medicine, Sema4, Caris, Guardant). Other vendors will be familiar to ordinarily skilled artisans. Genetic data may be provided directly to the knowledgebase, with genetic variants of interest being identified for processing in accordance with contents of the knowledgebase. Those genetic variants may then be stored separately in a database. A report may be prepared, summarizing findings regarding the genetic variants. Similar processes may be performed on identified mutations.

In an embodiment, the patient's individual genetic data may be mapped to the knowledgebase to determine which drugs and/or drug classes may be actionable for treatment response, treatment resistance, or treatment toxicity. Actionable results may be used to generate a PDF report comprising ranked treatment recommendations which is provided to the client, who could be a patient, provider, or other authorized person to receive medical information on behalf of the patient. Actionable results may alternatively be returned to a user interface with ranked recommendations, and a reviewer having appropriate cancer clinical pharmacology credentials may take into consideration the patient's history, or the reviewer's own clinical experience with the treatments and cancer types, and may accept the ranked recommendations, or may decide to remove, replace, or re-order one or more of the recommendations. In this embodiment, after the system makes its recommendations, a reviewer may curate these recommendations and use them.

Looking at this process in a little more detail, each gene may be mapped to a drug, and may be provided a ranking relative to other genes. In an embodiment, as reviewers re-order recommendations, the re-orderings may be tracked in a database which may or may not be part of the same knowledgebase discussed above.

In an embodiment, statistical methods employing machine learning may be used to train models and refine rankings over time. In an embodiment, recommendations and/or rankings may be provided based on characteristics of patients such as sex, cancer type, previous patient clinical histories, and other detected genes. In an embodiment, the statistical methods may include, but are not limited to Random Forest, Neural Networks, RankRLS, RankNet, LambdaRank, LambdaMART (LambdaMART being a combination of LambdaRank and Multiple Additive Regression Trees (MART)). Other suitable ranking algorithms will be familiar to ordinarily skilled artisans. Ranking quality can improve as the system learns patient drug recommendations from reviewers based on genetic variation.

Following is an exemplary algorithm in which the priority score of a recommendation is calculated using the following equation:

$$X = \frac{{\sum }_{i = 1}^{n} ( {A}_{i} x {B}_{i} ) * {C}_{i}}{n}$$

$$Score = \frac{X}{Z}$$

Where n equals the number of reports in the dataset used for calculating the ranks; A equals rank of the gene-drug recommendation for the ith report; B is the number of recommendations on the ith report; and C is a weighting factor of 1.2 if the ranking was considered high priority (i.e. in the impression section) of the report or 1.0 if not in the impression section. Z is the total number of times a gene-drug recommendation is mentioned across the n reports. A lower score would represent a higher predicted priority ranking for inclusion on a subsequent report.

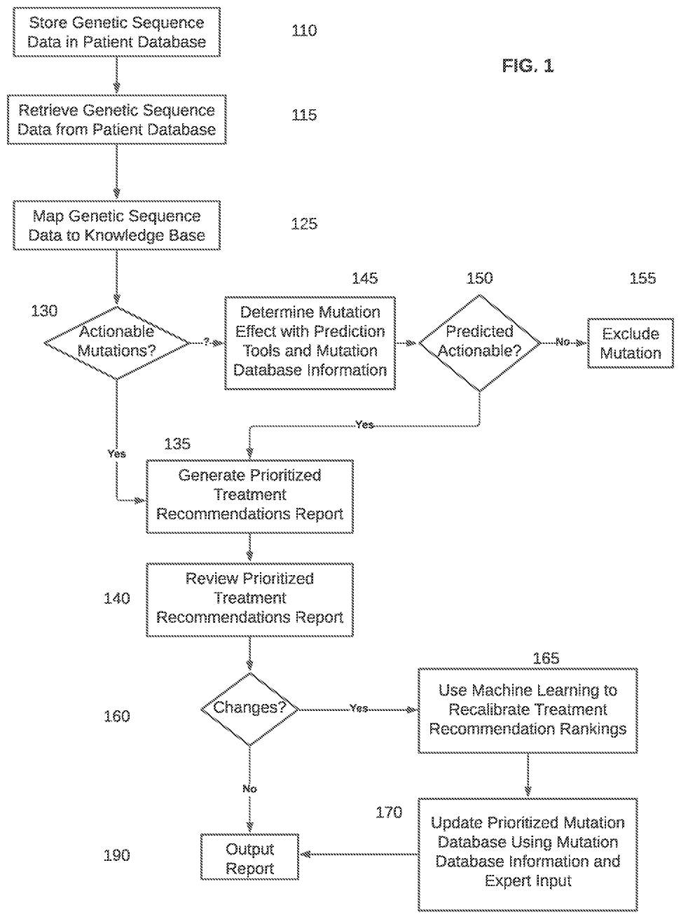

FIG. 1 is a high level flow chart of operation according to an embodiment. Initially, according to an embodiment, at 110, patient data, in the form of a genomic profile report or a variant call format (VCF) report is input to a patient database. At 115, genetic sequence data may be retrieved from the patient database and applied to one or more machine learning systems. At 125, the genetic sequence data is mapped to a knowledge base.

The genetic sequence data may fall into one of two categories. One category is actionable mutations. The other is variants of uncertain therapeutic consequence. At 130, if there are actionable mutations, then at 135 a prioritized treatment recommendation report is generated, setting forth a number of ranked treatment recommendations (in an embodiment, there may be one, two, three, four, five or more such recommendations, but a larger number of recommendations may be provided), and providing a recommended order of priority.

At 140 a doctor or other provider performs a clinical review of the prioritized treatment report. The doctor or other provider may have a different ordering of recommendations, or may even have one or more different recommendations to substitute for recommendation(s) in the report. At 160, if there are no changes as a result of the review, then at 190 a report is generated. If there are changes, then at 165 the prioritization is recalibrated via input to a machine learning system. At 170, a prioritized mutation/drug reference database may be updated. In an embodiment, the prioritized mutation/drug reference database may be compiled from one or more publicly available mutation databases, and appropriate expert knowledge may be applied to the database to provide the prioritization. Examples include the Clinical Pharmacogenetics Implementation Consortium (CPIC) database (https://cpicpgx.org/); the Pharmacogenomics Knowledge Base (PharmGKB) (https://www.ncbi.nlm.nih.gov/clinvar/); the ClinVar database; the cBioPortal database https://www.cbioportal.org/; the Oncology Knowledgebase (OncoKB) (https://www.oncokb.org); the Cancer Genome Atlas (TCGA) (https://www.cancer.gov/about⋅nci/organization/ccg/research/structural-genomics/tcga); and the Catalog of Somatic Mutations in Cancer (COSMIC) database (https://cancer.sanger.ac.uk/cosmic). The expert knowledge may be in the form of review of the contents of the database by one or more providers or precision medicine experts.

Once the prioritized mutation/drug reference database is updated using the results of the clinical review, at 190 a report is output and may be sent to a doctor, hospital, or other customer.

It should be noted that the updating could occur either before or after the report is generated. The timing of the updating is not critical to the generation of the report.

The just-described process is not the only path to generating a prioritized treatment recommendations report. Returning to 130, if variants of unknown significance are provided, further processing on those variants may be performed. Accordingly, at 145 prediction tools and mutation database information are used to determine a mutation effect. In an embodiment, prediction tools, which may involve a machine learning system, may be applied to data in publicly available mutation databases of a type similar to those mentioned above, to obtain a mutation effect determination. For example, a variant may be reported in the ClinVar database to be “pathogenic” or “likely pathogenic”. Alternatively or additionally, the variant could be reported to be a truncating mutation, fusion, missense, or other mutation type that is deemed likely to confer a functional effect on the gene/protein in reference in a database such as COSMIC. Different databases or knowledge bases may experience different responses or changes to reported variants.

At 150, if, as a result of this determination, a mutation is predicted to be actionable, then flow proceeds to 135, and a prioritized treatment recommendations report is generated, similarly to the flow proceeding from 130. At this point, a mutation that is predicted to be actionable is treated in the same manner as one that was determined previously to be actionable. On the other hand, if a mutation predicted to be non-actionable, then at 155 that mutation is excluded from further consideration.

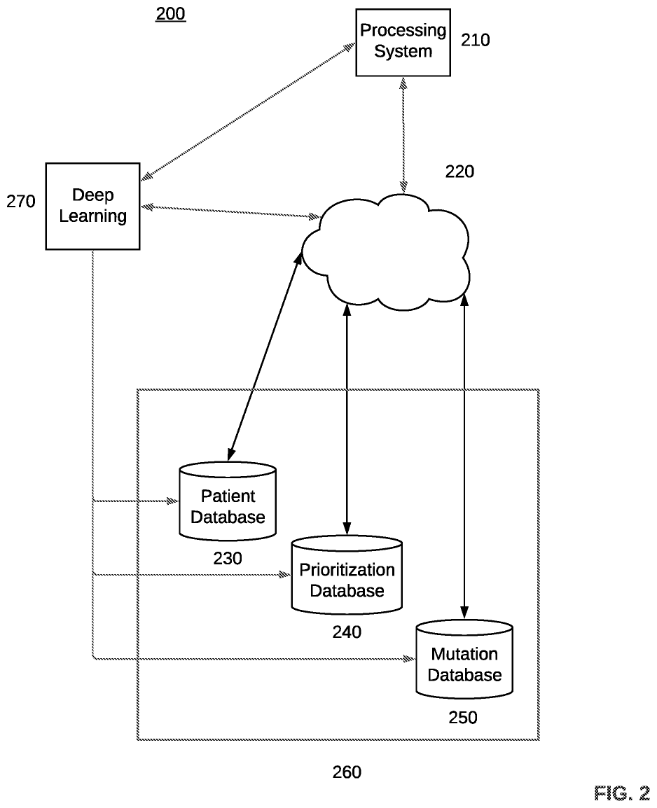

FIG. 2 is a high level block diagram of a system 200 according to an embodiment. Processing system 210 may include one or more processors, one or more storage devices intended for non-volatile non-transitory storage, and one or more memory devices, which may be volatile memory for transitory storage, but which also may include non-volatile memory for non-transitory storage. Network 220 may be an internal network, or may be a cloud-based network. Network 220 connects processing system 210 to one or more of a patient database 230, a prioritization database 240, and a mutation database 250. Some or all of these databases may be merged into a single database, and/or may form the above-referenced knowledge base, denoted 260.

Machine learning system 270 may include one or more processors, one or more storage devices, and one or more memory devices, and may communicate with any or all of processing system 210, databases 230-250 (or knowledge base 260) via network 220. In an embodiment, system 270 may include a plurality of such systems.

In an embodiment, the processors in machine learning system 270 may be graphics processing units (GPUs) or central processing units (CPUs), which can lend themselves to neural network structures or other learning frameworks. In an embodiment, a neural network forming part of machine learning system 270 may include any of a plurality of types of neural networks, including convolutional neural networks (CNN), deep or fully convolutional neural networks (DCNN, FCNN), deep learning neural networks (DNN), deep belief networks (DBN), and others with which ordinarily skilled artisans will be familiar. In an embodiment, machine learning system 270 may be a multiple instance learning (MIL) system. In some nomenclature, deep learning systems are distinguished from artificial intelligence (AI) or machine learning (ML) systems or MIL systems in various ways. For purposes of the present discussion, any or all of deep learning, AI, ML, and MIL systems may provide the necessary structure to accomplish one or more inventive goals.

### Sources of Knowledge Informing the Knowledgebase

Sources of knowledge informing the knowledgebase can be from any source that reveals human genetic mutations including somatic and germline mutations. Knowledge sources include e.g., The Cancer Genome Atlas (TCGA) Research Network (see e.g., Weinstein, J. N. et al. (2013) Nat. Genetics 45(10):113-1120), patient studies that analyze the relationship of human mutations and cancer (see e.g., Nadauld L. D., et al. Molecular profiling of gastric cancer: toward personalized cancer medicine. J Clin Oncol. 2013; 31:838-839), as well as Clinical Pharmacogenetics Implementation Consortium (CPIC) database (https://cpicpgx.org/); the Pharmacogenomics Knowledge Base (PharmGKB) (https://www.ncbi.nlm.nih.gov/clinvar/); the ClinVar database; the cBioPortal database https://www.cbioportal.org/; the Cancer Genome Atlas (TCGA) (https://www.cancer.gov/about⋅nci/organization/ccg/research/structural-genomics/tcga); the Oncology Knowledgebase (OncoKB) (https://www.oncokb.org); and the Catalog of Somatic Mutations in Cancer (COSMIC) database (https://cancer.sanger.ac.uk/cosmic).

### Patient Data

The machine learning systems and knowledgebase provided herein give healthcare providers the ability to utilize individual genetic information to determine if a gene or the region that regulates a gene comprises mutations/variants that are linked to a disorder and if so, to recommend a therapeutic regimen to treat the corresponding disease or disorder. Typically, patient data is in the form of DNA sequence data which may be obtained from any known sequencing method, e.g., whole genome sequencing.

### Whole Genome Sequencing

Whole genome sequencing (WGS) provides the clinician a comprehensive view of a patient's entire set of genetic material. A clinician can order or perform whole genome sequencing to determine a patient's individual genetic make-up. Information obtained from WGS may reveal e.g., single nucleotide variants (SNVs), copy number changes, insertions, deletions, fusions, and/or structural variants that are associated with cancer or genetic disease or which may be associated with a patient's response to particular drugs or therapy.

DNA samples are typically obtained from any biological sample containing a full copy of genomic DNA. For example patient sample may be taken from tumor tissue, blood, saliva, epithelial cells, bone marrow, hair follicle, etc.

Samples are subjected to sequencing utilizing any technique known in the art e.g., utilizing Illumina dye sequencing (see e.g., Meyer M, Kircher M (J 2010). “Illumina sequencing library preparation for highly multiplexed target capture and sequencing” Cold Spring Harbor Protocols. 2010 (6): pdb.prot5448. doi: 10.1101/pdb.prot5448), pyrosequencing, Single Molecule Real time (SMRT) sequencing (see e.g., Levene M J, et al. (2003) Science. 299 (5607): 682-686), nanopore technology (see e.g., Liu Z, et al. Journal of Nanomaterials. 2016:1-13), etc.

In some embodiments, genetic data may be obtained from an individual cancer. Thus, in some embodiments, samples obtained from a cancer biopsy are subjected to cancer whole-genome sequencing (WGS) for example utilizing with next-generation sequencing (NGS).

Genomic data obtained from a patient sample is then downloaded to a patient database (110) where it is available to be retrieved (115) for use in the disclosed machine learning system (120) for mapping to the knowledgebase (125).

While certain embodiments of the present invention have been shown and described herein, it will be obvious to ordinarily skilled artisans that these embodiments are merely exemplary. Numerous variations, changes, and substitutions will occur to ordinarily skilled artisans within the scope and spirit of the invention. Various alternatives to the described embodiments may be employed. Accordingly, the invention should be considered as limited only by the scope of the following claims, and that methods and structures within the scope of these claims and their equivalents are covered.

### EXAMPLES

### Example 1

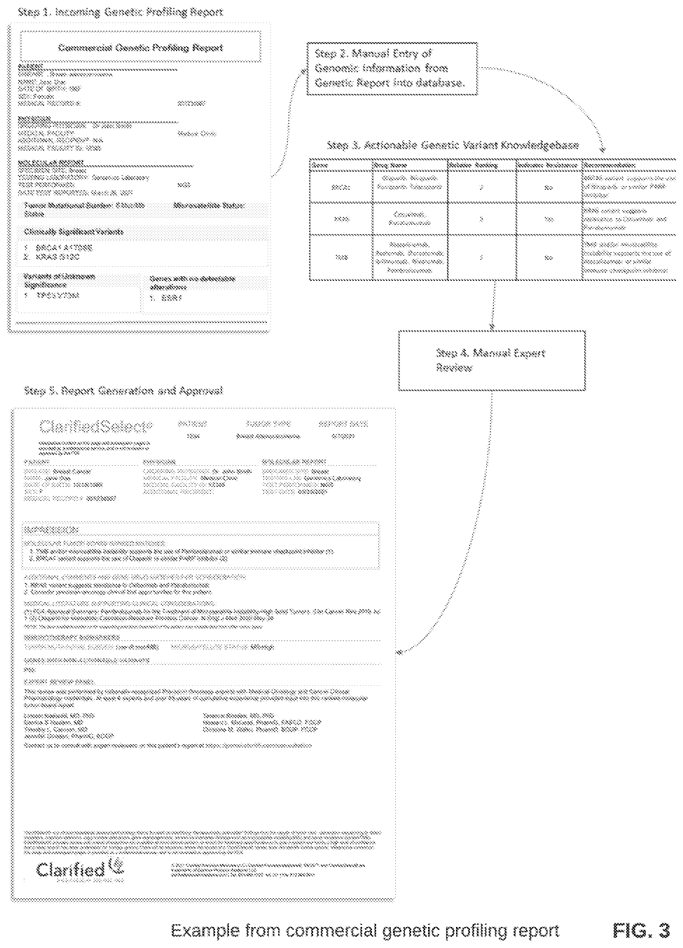

FIG. 3 illustrates a representative genomic data analysis and generation of a treatment recommendation for PARP and immune checkpoint inhibitors based on the receipt of a commercial genetic profiling report for a breast cancer specimen in a patient, inclusive of specific literature citations supportive of same.

### Example 2

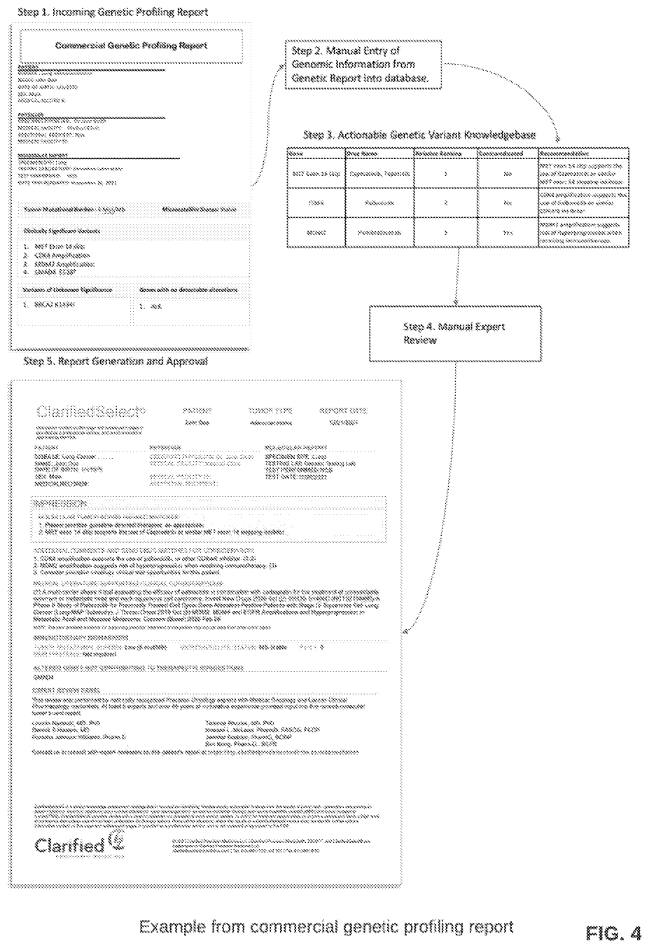

FIG. 4 illustrates a representative genomic data analysis and generation of a treatment recommendation for MET inhibitors based on the receipt of a commercial genetic profiling report for a lung cancer specimen in a patient, inclusive of specific literature citations supportive of same.

### Example 3

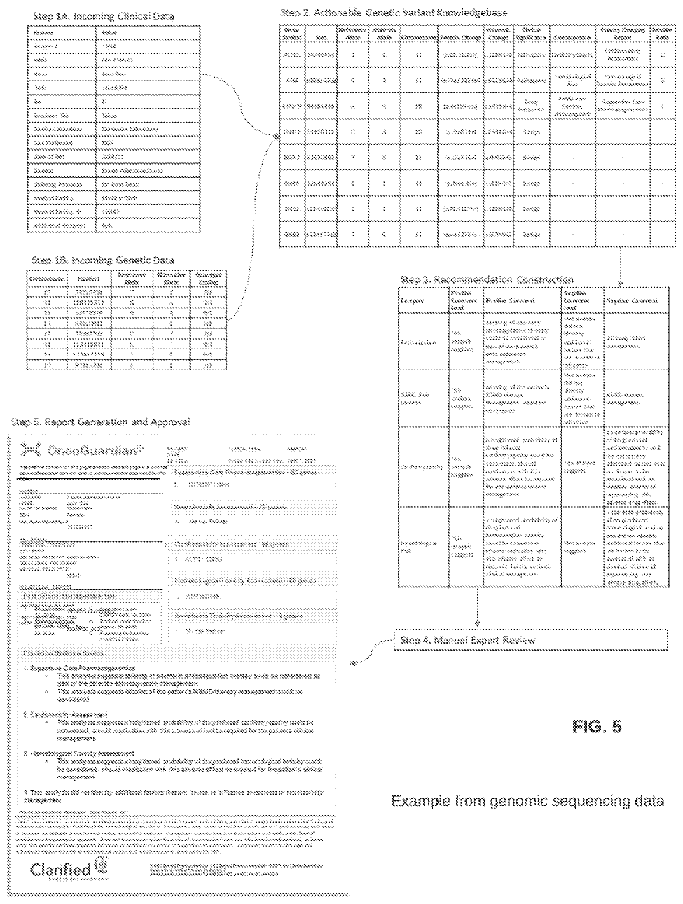

FIG. 5 illustrates a representative genomic data analysis and generation of a treatment recommendation relating to potential cardiotoxicity, hematological toxicity, and supportive care pharmacogenomics based on receipt of genomic sequencing data from a patient saliva sample.

What is claimed is:

1. A method of generating a prioritized precision treatment recommendation for a patient, comprising:
receiving genetic sequence data for said patient comprising at least one genetic mutation; optionally wherein said at least one genetic mutation is identified after receipt;
applying said patient-specific genetic sequence data comprising said at least one genetic mutation identified in one or more samples of a patient, to a machine learning system trained on a knowledgebase comprising a plurality of genetic mutations across a plurality of genes to map said genetic sequence data to said knowledgebase;
said knowledgebase mapping said plurality of genetic mutations to efficacy profiles for therapeutic regimens for the disease, and/or further mapping said genetic mutations to drug-induced toxicities selected from the group consisting of cardiotoxicity, neurotoxicity, hematological toxicity, and anesthesia toxicity;
determining, by the machine learning system, a plurality of therapeutic regimens, which may be actionable as a treatment recommendation for said disease for said patient based on one or more of treatment response, treatment resistance, or treatment toxicity; and
prioritizing, by said machine learning system, the therapeutic regimens to provide a plurality of ranked treatment recommendations for said disease for said patient as determined by the machine learning system.

2. The method of claim 1, wherein said at least one genetic mutation is somatic or germline.

3. The method of claim 1, wherein each said at least one genetic mutation is mapped to a drug and provided a ranking relative to other genes.

4. The method of claim 1, further comprising reviewing, by an expert, the plurality of ranked treatment recommendations and, responsive to a determination that the ranked treatment recommendations should be reordered or changed, providing a revised set of ranked treatment recommendations; optionally wherein the knowledgebase is updated based on the revised set of ranked treatment recommendations.

5. The method of claim 1, further comprising communicating the plurality of ranked treatment recommendations for said disease for said patient to the patient and/or to the patient's caregiver.

6. The method of claim 4, further comprising communicating the revised set of ranked treatment recommendations for said disease for said patient to the patient and/or to the patient's caregiver.

7. The method of claim 1, wherein the patient-specific genetic sequence data comprises sequence variants with known functional effects or sequence variants with unknown clinical significance.

8. The method of claim 1, wherein the ranked treatment recommendations comprise off-label uses and/or clinical trials.

9. The method of claim 1, wherein the ranked treatment recommendations further comprise supporting literature citations.

10. The method of claim 1, wherein said disease is cancer, and the patient-specific genetic sequence comprises tumor panel sequencing data from at least one tumor sample from said patient, and wherein the knowledge base comprises a plurality of genetic mutations across a plurality of genes in a plurality of tumor types from a plurality of individuals and a plurality of treatments.

11. A method of treating a disease in a patient in need thereof, comprising:
receiving genetic sequence data for said patient comprising at least one genetic mutation; optionally wherein said at least one genetic mutation is identified after receipt;
applying said patient-specific genetic sequence data comprising said at least one genetic mutation identified in one or more samples of a patient, to a machine learning system trained on a knowledgebase comprising a plurality of genetic mutations across a plurality of genes to map said genetic sequence data to said knowledgebase;
said knowledgebase mapping said plurality of genetic mutations to efficacy profiles for therapeutic regimens for the disease, and/or further mapping said genetic mutations to drug-induced toxicities selected from the group consisting of cardiotoxicity, neurotoxicity, hematological toxicity, and anesthesia toxicity;
determining, by the machine learning system, a plurality of therapeutic regimens, which may be actionable as a treatment recommendation for said disease for said patient based on one or more of treatment response, treatment resistance, or treatment toxicity;
prioritizing, by said machine learning system, the therapeutic regimens to provide a plurality of ranked treatment recommendations for said disease for said patient as determined by the machine learning system;
communicating the ranked treatment recommendations for said disease for said patient to the patient's caregiver; and
administering, by said caregiver, at least one of the ranked treatment recommendations.

12. The method of claim 11, wherein said at least one genetic mutation is somatic or germline.

13. The method of claim 11, wherein each said at least one genetic mutation is mapped to a drug and provided a ranking relative to other genes.

14. The method of claim 11, further comprising reviewing, by an expert, the plurality of ranked treatment recommendations and, responsive to a determination that the ranked treatment recommendations should be reordered or changed, providing a revised set of ranked treatment recommendations; and said communicating comprises communicating the revised set of ranked treatment recommendations for said disease for said patient to the patient and/or to the patient's caregiver; optionally wherein the knowledgebase is updated based on the revised set of ranked treatment recommendations.

15. The method of claim 11, wherein the patient-specific genetic sequence data comprises sequence variants with known functional effects or sequence variants with unknown clinical significance.

16. The method of claim 11, wherein the ranked treatment recommendations comprise off-label uses and/or clinical trials.

17. The method of claim 11, wherein the ranked treatment recommendations further comprise supporting literature citations.

18. The method of claim 11, wherein said disease is cancer, and the patient-specific genetic sequence comprises tumor panel sequencing data from at least one tumor sample from said patient, and wherein the knowledge base comprises a plurality of genetic mutations across a plurality of genes in a plurality of tumor types from a plurality of individuals and a plurality of treatments.
